(19) United States
(12) Patent Application Publication
(10) Pub. No.: US 2024/0319181 A1
(43) Pub. Date: Sep. 26, 2024

(54) CHEMILUMINESCENT LATERAL FLOW IMMUNOASSAY METHOD

(71) Applicant: Peking University, Beijing (CN)

(72) Inventor: Yue CUI, Beijing (CN)

(73) Assignee: Peking University, Beijing (CN)

(21) Appl. No.: 18/391,740

(22) Filed: Dec. 21, 2023

(30) Foreign Application Priority Data
Mar. 21, 2023 (CN) 202310273099.1

(51) Int. Cl. G01N 33/543; G01N 21/76; G01N 33/532

(52) U.S. Cl. CPC G01N 33/54388; G01N 21/76; G01N 33/532; G01N 2333/902

(57) ABSTRACT

A chemiluminescent lateral flow immunoassay method is provided for reducing cross reactions and false positives when detecting protein analytes. The detection method involves a lateral flow immunoassay strip, the strip includes a sample pad, a conjugate pad, a nitrocellulose membrane, and an absorbent pad. The capture antibody of the analyte on the nitrocellulose membrane is used as the test line, and the IgG antibody is used as the control line. The lateral flow immunoassay is performed on the strip by the Au nanoparticle-antibody-horseradish peroxidase-polyethylene glycol (AuNP-Ab-RP-PEG) conjugate to detect the specific analyte. During the detection, the conjugate is prepared in the strip and then added to the analyte for detection, or the conjugate is mixed with the analyte and then added to the strip for detection.

### CROSS REFERENCE TO THE RELATED APPLICATIONS

This application is based upon and claims priority to Chinese Patent Application No. 202310273099.1, filed on Mar. 21, 2023, the entire contents of which are incorporated herein by reference.

### TECHNICAL FIELD

The invention relates to the field of flow immunoassay technology, in particular to a chemiluminescent lateral flow immunoassay method.

### BACKGROUND

Lateral flow immunoassay (LFIA) is invented in 1956 and has been receiving increasing attentions for its widespread use in various fields such as medical and health care, food safety, environmental monitoring, and so on. The development of LFIA strips is particularly important for the diagnosis of clinical protein biomarkers, which further realizes the application of LFIA in the home setting. Due to the low concentration of the clinical biomarker in biofluids, an ultrasensitive detection is highly desired. For example, acute myocardial infarction (AMI) is the first of the world's top ten deadly diseases. cTnI is considered to be the ‘golden standard biomarker’ for the early diagnosis of AMI because the concentration changes in cTnI are directly related to the different development stages of AMI. However, cTnI can be as low as 0.05 ng/ml in human serum under normal circumstances. Therefore, during the first 3 h of AMI, the concentration of cTnI is too low to be detected. More importantly, the concentration of cTnI is dynamic depending on the different stages of AMI, highly sensitive and specific cTnI detection is necessary for AMI patients.

Enhancing the sensitivity of lateral flow immunoassay is crucial for the detection of many biomarkers. In recent years, people have been working to develop new strips with high sensitivity, mainly by conjugating antigens or antibodies to nanomaterials, such as gold nanoparticles (AuNPs), magnetic nanoparticles, quantum dots, and fluorescent microspheres. Among these methods, the conjugating antibodies and horseradish peroxidase (HRP) to AuNPs can produce effective and ultrasensitive chemiluminescent immunoassays (CL-LFIAs). The main reasons for the false positive error are the nonspecific adsorption between antigen and antibody and the nonspecific adsorption between protein and nitrocellulose membrane. Therefore, a method to minimize the cross reactions to achieve its high accuracy is urgently required by LFIA.

In order to reduce false positive errors and improve detection accuracy, one of the effective methods is to remove the Fc region in antibodies, which is the main cause of nonspecificity. In addition, biotinylated antibodies can also be applied. The antigen-antibody interaction is converted to an avidin-biotin interaction, and the specific immobilization is greatly enhanced, thereby improving the sensitivity and specificity. However, both methods involve changes in antibody structure, which may further affect the immobilization reactivity. Another method is an ELISA method based on a two-phase water-based system, which fixes the detection antibody in a certain area of the water environment, the antibody is prevented from interacting, and the cross-reactivity is eliminated. However, this method requires an accurate micropipette, thus the method is not suitable for large-scale and rapid detection. The hydrophobic cadmium-based quantum dots with enhanced dispersibility are studied as probes for the detection of SARS-CoV-2, which can minimize cross reactions and improve accuracy. Other nanoparticles, such as AuNPs, upconverting NPs, and magnetic iron-oxide NPs, are also used to detect biomarkers with high accuracy and sensitivity. For example, antibody-functionalized magnetic iron-oxide nanoparticles are used to reduce nonspecific signals and detect dual markers with high accuracy. By using the secondary antibody bound by AuNPs, the multiple detection of three hormones is realized by detecting the SPR signal of AuNPs, and there is no cross reaction. However, these methods involve the synthesis of nanoparticles and the procedure is complicated. In addition, the aggregation of these NPs may also lead to wrong signals due to the interaction of nanoparticles. The sensitivity of multiplex immunoassay is increased by nearly 1000 times by using ultrasonic standing wave technology to disperse AuNPs to eliminate the nonspecific interaction between antibodies and AuNPs. However, the ultrasonic equipment may not satisfy the point-of-care detection.

The key factor to reduce false positive errors in nanoparticle-based immunoassay is to improve the dispersion and reduce the aggregation of nanoparticles. Current studies have shown that PEGylation of nanoparticles can reduce the adsorption of proteins and cells, and with the increase of molecular weight and density of PEG, the efficiency will be higher. However, the PEGylation of AuNPs in chemiluminescent LFIA has not been studied until now.

### SUMMARY

The purpose of this invention is to provide a chemiluminescent lateral flow immunoassay method, the PEGylation of AuNPs can effectively reduce protein adsorption and nonspecific reaction, so cross reactions can be minimized, and further, the accuracy and sensitivity of detection are improved.

In order to achieve the above purpose, the invention provides a chemiluminescent lateral flow immunoassay method, the method is used to reduce cross reaction and false positive in a detection of protein analyte, using an Au nanoparticle-antibody-horseradish peroxidase-polyethylene glycol (AuNP-Ab-HRP-PEG) conjugate to perform a lateral flow immunoassay on a strip for a detection of specific analyte.

Preferably, a preparation method for the AuNP-Ab-HRP-PEG conjugate includes the following steps:
S1, conjugating a detection antibody of an HRP-conjugated analyte to AuNPs by a physisorption method: adding a sodium carbonate solution to an AuNP solution, and then adding an HRP-labeled detection antibody and then stirring gently on a rotator;
S2, linking mPEG covalently to the AuNPs by Au—S bonds: adding an mPEG solution to a mixture in step S1 and stirring gently on the rotator for further incubation;
S3, incubating a BSA solution with the AuNPs and stirring gently on the rotator to block additional reaction sites on AuNPs, then centrifuging an incubation product, and then adding a borate buffered solution of pH 8-9 to obtain the Au nanoparticle-antibody-horseradish peroxidase-polyethylene glycol (AuNP-Ab-HRP-PEG) conjugate;
S4, suspending the AuNP-Ab-HRP-PEG conjugate obtained by step S3 in a recovery solution containing 0.5-10% BSA, 1-10% sucrose and 0.01-1% tween20 borate buffered solution, and storing the conjugate for further use.

Preferably, the strip includes a backing plate, a sample pad, a conjugate pad, a nitrocellulose membrane, and an absorbent pad; the sample pad, the conjugate pad, the nitrocellulose membrane, and the absorbent pad are arranged in turn to be immobilized onto the surface of the backing plate; the surface of the nitrocellulose membrane is equipped with a test line and a control line, the test line is formed by immobilizing capture antibody onto a surface of the nitrocellulose membrane, and the control line is formed by immobilizing IgG antibody onto the surface of the nitrocellulose membrane; the conjugate pad contains the AuNP-Ab-HRP-PEG conjugate.

Preferably, a preparation method for the strip includes the following steps:
a, immobilizing capture antibody on the nitrocellulose membrane to form the test line, and then immobilizing IgG antibodies on the nitrocellulose membrane to form the control line;
b, dispensing the prepared the AuNP-Ab-HRP-PEG conjugate onto the conjugate pad;
c, overlapping the sample pad, conjugate pad, nitrocellulose membrane, and absorbent pad to the backing plate in turn, and then cutting the backing plate into strips with a width of 1-10 mm, and placing the strips in a dry environment at room temperature for further use.

The above chemiluminescent lateral flow immunoassay method includes the following steps:
adding an analyte to the sample pad or conjugate pad of the strip, the conjugate pad of the strip containing the AuNP-Ab-HRP-PEG conjugate, and then waiting for 0.5-10 minutes, adding a chemiluminescence substrate, taking a photo for recording after a luminescence.

Preferably, the strip includes the backing plate, the sample pad, the conjugate pad, the nitrocellulose membrane and the absorbent pad, the sample pad, the conjugate pad, the nitrocellulose membrane, and the absorbent pad are arranged in turn to immobilize the surface of the backing plate; the surface of the nitrocellulose membrane is equipped with the test line and the control line, the test line is formed by immobilizing capture antibodies to a surface of the nitrocellulose membrane, and the control line is formed by immobilizing IgG antibodies to the surface of the nitrocellulose membrane; the conjugate pad doesn't contain the AuNP-Ab-HRP-PEG conjugate.

Preferably, the preparation method for the strip includes the following steps:
a, immobilizing the capture antibody of the analyte on the nitrocellulose membrane to form the test line, and then immobilizing IgG antibodies on the nitrocellulose membrane to form the control line;
b, overlapping the sample pad, conjugate pad, nitrocellulose membrane, and absorbent pad to the backing plate in turn, and then cutting into strips with a width of 1-10 mm, and placing the strips in a dry environment at room temperature.

The chemiluminescent lateral flow immunoassay method mentioned above includes the following steps:
mixing the analyte with the AuNP-Ab-HRP-PEG conjugate to form a mixed solution, and then adding the mixed solution to the sample pad or conjugate pad of the strip, and then waiting for 0.5-10 minutes, adding a chemiluminescent substrate, taking a photo for recording after the luminescence.

The advantages and positive effects of the chemiluminescent lateral flow immunoassay method described in the invention are as follows:

1. In this invention, the PEGylation of AuNPs can effectively reduce protein adsorption and nonspecific reaction, minimize cross reaction, and further improve the accuracy and sensitivity of protein analyte detection.

2. In this invention, AuNPs are functionalized by HRP-conjugated detection antibodies, and mPEG-SH is conjugated to AuNPs through Au—S covalent bonds, in the presence of the chemiluminescent substrate of the enzyme, HRP can effectively catalyze the enzymatic reaction and emit a strong chemiluminescence, which can be easily detected by a mobile phone with a CMOS camera.

3. The AuNP-Ab-HRP-PEG conjugate in this invention has been studied for the chemiluminescent lateral flow tests of cTnI, and it has also been studied for the detection of different and various analytes. Through PEG functionalization of AuNPs, higher sensitivity and accuracy can be obtained for portable immunoassay devices, the detection time is short and the procedure is simple. It can be used not only for lateral flow chemiluminescence detection, but also for other detections, such as nanoparticle-based color detection, and other detection methods.

The following is a further detailed description of the technical solution of the invention through drawings and an embodiment.

### BRIEF DESCRIPTION OF THE DRAWINGS

FIG. 1 is a schematic diagram of the preparation of the AuNP-Ab-RP-PEG conjugate in the embodiment of the invention;

FIGS. 2A-2C are illustrations of PEGylated AuNPs in the embodiment of the invention to improve the performance of the CL-LFIA biosensor;

FIGS. 3A-3D are dynamic light scattering results of AuNPs, AuNP-PEG, AuNP-PEG-Ab, and AuNP-Ab conjugates in the embodiment of the invention;

FIGS. 4A-4D are a UV-visible spectrum and an FTIR spectrum of different conjugates in the embodiment of the invention, where FIG. 4A is a UV-visible spectrum of different conjugates, FIG. 4B is an amplified absorbance curve of 500 nm-600 nm wavelength in FIG. 4A, FIG. 4C is an FTIR spectrum of the AuNP-PEG conjugate, and FIG. 4D is an amplified absorbance curve of 2000 cm⁻¹-1200 cm⁻¹ wavelength in FIG. 4C;

FIG. 5 is an illustration diagram of the AuNP-Ab-HRP-PEG conjugate applied to CL-LFIA detection in the embodiment of the invention.

FIGS. 6A-6D are schematic diagrams of the influence of different parameters on the sensitivity and specificity of the LFIR strip in the CL-LFIA test optimization of the embodiment of the invention; where FIG. 6A is a schematic diagram of the influence of the PEG concentration on the signal intensity of the test line and the control line, the concentration of the analyzer is 0, FIG. 6B is a schematic diagram of the influence of the PEG concentration on the signal intensity of the test line and the control line, the concentration of the analyzer is 100 ng·mL⁻¹, FIG. 6C is a schematic diagram of the influence of the concentration of conjugated antibody on the CL-LFIA detection, and FIG. 6D is a schematic diagram of the influence of optical signal intensity on the change of acquisition time.

FIGS. 7A-7C are determination result diagrams of cTnI in PBS buffer based on the optimization of the CL-LFIA test in the embodiment of the invention, where FIG. 7A is an average intensity of cTnI concentration from 10 pg·mL⁻¹ to 100 ng·mL⁻¹, FIG. 7B is a calibration curve diagram of cTnI, and FIG. 7C is a sensitivity detection diagram.

### MARKS OF ATTACHED FIGURES

1, backing plate; 2, sample pad; 3, conjugate pad; 4. nitrocellulose membrane; 5, absorbent pad.

### DETAILED DESCRIPTION OF THE EMBODIMENTS

The following is a further explanation of the technical solution of the invention through drawings and an embodiment. Unless otherwise defined, the technical terms or scientific terms used in the invention should be understood by people with general skills in the field to which the invention belongs.

All experimental materials are conventional commercially available products.

### EMBODIMENT

The preparation method for the AuNP-Ab-HRP-PEG conjugate includes the following steps:
S1, an RP-conjugated anti-cTnI detection antibody of an RP-conjugated analyte is conjugated to AuNPs by physisorption method: 20 μL of 0.02 M sodium carbonate solution is added to 0.5 mL AuNP solution, and then adding 12.5 μL of 1 mg·mL¹ HRP-labeled detection antibody and then stirring gently on a rotator for 30 minutes;
S2, an mPEG covalently is linked to AuNPs by an Au—S bond: 20 μL of 5% mPEG solution is added to a mixture in step S1 and is stirred gently on the rotator, and further the mixture is incubated for 30 minutes;
S3, 50 μL of 10% BSA solution is incubated with AuNPs for 30 minutes, and the product is stirred gently on the rotator to block additional reaction sites on AuNPs, then the incubation product is centrifuged at 10000 rpm for 3 times, a borate buffered solution of pH8-9 is added after 12 minutes to obtain the AuNP-Ab-RP-PEG conjugate;
S4, the AuNP-Ab-RP-PEG conjugate obtained by step S3 is suspended in 200 μL recovery solution and the conjugate is stored at 4° C. for further use.

Where the pH of the borate buffered solution is 8.5, and the recovery solution is a borate buffered solution containing 1% BSA, 2% sucrose, and 0.05% tween20.

The AuNP-Ab-RP conjugate without PEGylation is prepared by the same method as above, except that mPEG solution is not added in step S2.

After the successful preparations of two conjugates, different characterization methods are carried out:

The hydrate particle size of different conjugates is measured by a laser light scattering spectrometer, the UV-Vis spectra of different conjugates is obtained by using Shimadzu UV3600Plus UV-Vis-NIR spectrophotometer, the Fourier transform infrared (FTIR) spectra of the conjugates is obtained by using an FTIR spectrophotometer.

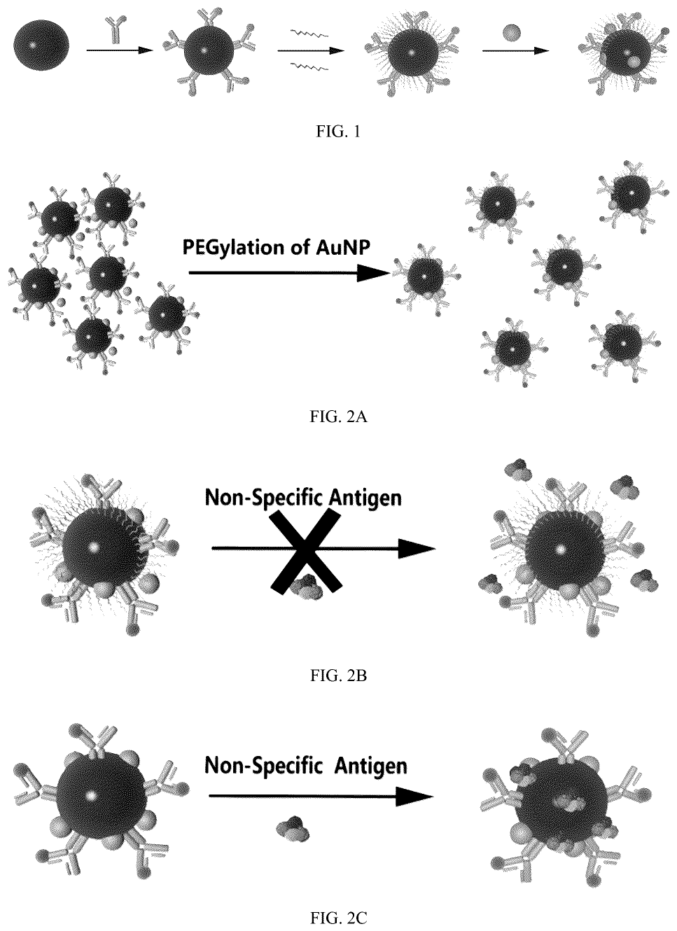

The principle of chemiluminescent lateral flow immunoassay (CL-LIFA) using the AuNP-Ab-HRP-PEG conjugate is as follows: first, the HRP-conjugated antibody (Ab) is conjugated to AuNPs by the physisorption method, and then the product is further conjugated to mPEG-SH by Au—S covalent bond, as shown in FIG. 1.

As shown in FIGS. 2A-2C, after PEGylation of AuNPs (as shown in FIG. 2A), the nonspecific adsorption of other antigens on AuNPs is significantly reduced (shown in FIG. 2B) compared with AuNPs without PEG functionalization (as shown in FIG. 2C).

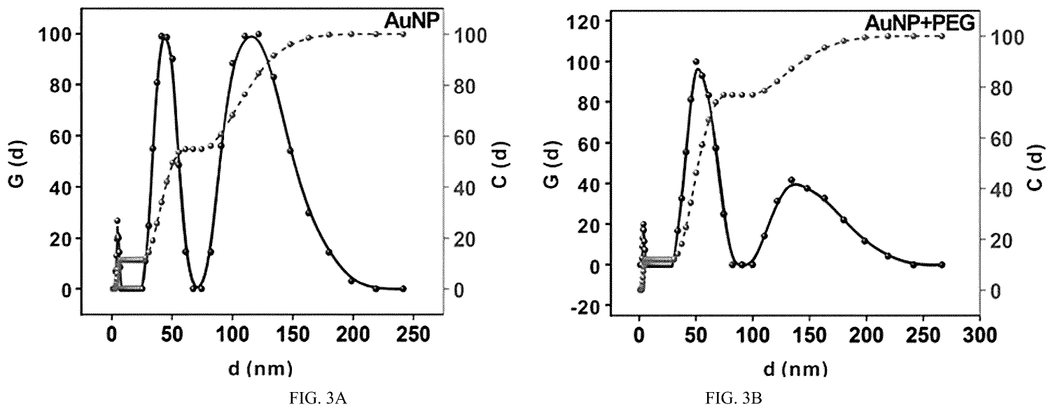

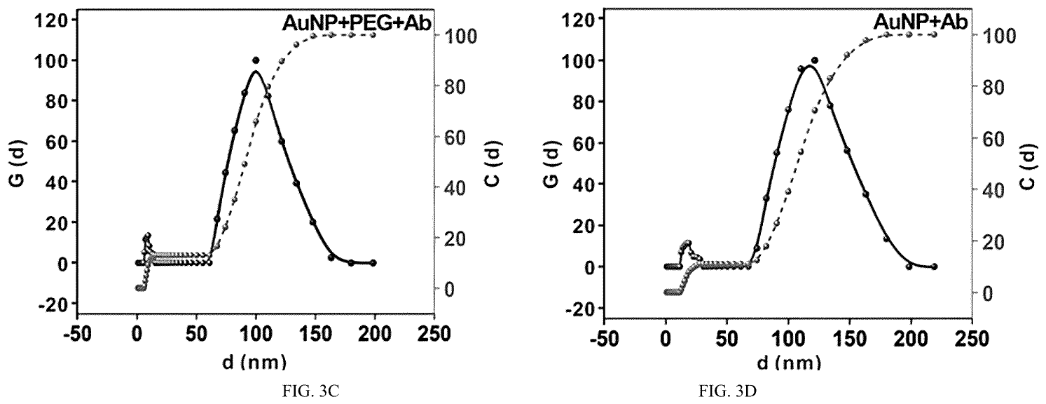

The characteristics of the AuNP-Ab-RP-PEG conjugate: The hydrodynamic diameter distribution of different conjugates is evaluated by dynamic light scattering (DLS), including AuNPs conjugate (as shown in FIG. 3A), AuNP-PEG conjugate (as shown in FIG. 3B), AuNP-PEG-Ab conjugate (as shown in FIG. 3C) and AuNP-Ab conjugate (as shown in FIG. 3D), where the left ordinate represents the difference distribution of conjugate diameter, and the right ordinate represents the cumulative distribution of conjugate diameter. The difference distribution line in FIG. 3A shows two obvious peaks, mainly due to the aggregation of some AuNPs in the PBS buffer, and the addition of PEG in the AuNP solution can make AuNPs more dispersed in the PBS buffer. The comparison of FIG. 3A with FIG. 3B and the comparison of FIG. 3C with FIG. 3D show that the number of aggregated nanoparticles is reduced, because the average diameter after PEGylation is significantly reduced. The main reason why PEGylation can prevent nanoparticle aggregation is that the PEG layer covered on the nanoparticles can reduce nonspecific interactions.

Comparing the AuNP curve of FIG. 3A with the AuNP-Ab curve of FIG. 3D, there is only one peak in the difference distribution curve of FIG. 3D, which indicates the conjugation between antibody and AuNP. In addition, as shown in Table 1, the hydrodynamic diameter of AuNP increases significantly from 51.79 nm to 86.64 nm after the addition of antibody, indicating that the antibody was successfully bound to AuNP. As shown in Table 1, after PEGylation, the average hydrodynamic diameter of AuNP decreases from 51.79 nm to 50.96 nm, indicating that the colloidal gold solution becomes more dispersed. As shown in Table 1, after PEGylation, the average hydrodynamic diameter of AuNP-Ab decreases from 86.64 nm to 71.90 nm, indicating that the aggregation of AuNP-Ab conjugates is also inhibited. The comprehensive experimental results show that the addition of PEG reduces the average diameter of the particles and increases the diffusion coefficient, indicating that the number of aggregated AuNPs decreases.

TABLE 1
Dynamic light scattering results of
different conjugates hydrodynamic

| | | | Diffusion |
|---|---|---|---|
| | Mean hydrodynamic | | coefficient/ |
| Solution | diameter/(nm) | Polydispersity | (cm²/s) |
| AuNP | 51.79 | 0.301 | 9.475e−08 |
| AuNP-PEG | 50.96 | 0.277 | 9.630e−08 |
| AuNP-PEG-Ab | 71.90 | 0.303 | 6.825e−08 |
| AuNP-Ab | 86.64 | 0.284 | 5.664e−08 |

According to the results of dynamic light scattering, the mechanism of PEGylated AuNPs improving the performance of CL-LFIA can be obtained. Considering that there are many different proteins and molecules in blood or other solutions, the PEG-free conjugates may adsorb nonspecific antigens by electrostatic attraction, as shown in FIG. 2B; because there is enough space on the surface of the AuNPs, the additional active sites are occupied by mPEG-SH after PEGylation, which improves the steric hindrance between AuNP and nonspecific antigens, as shown in FIGS. 2A-2C. Therefore, the reduction of nonspecific adsorption will improve the specificity and accuracy of CL-LFIA.

In addition, after the addition of PEG, the aggregation of AuNPs tends to stay on the test line, which usually leads to a false positive line of LFIA. However, PEG on the surface of AuNPs may reduce the electrostatic attraction between nanoparticles, which may prevent the adsorption between AuNPs. As shown in FIG. 2A, the solution will be more dispersed, which has been confirmed by the results of DLS.

The AuNP-Ab-HRP-PEG conjugate is characterized by a UV-visible spectrophotometer and an FTIR spectrophotometer:

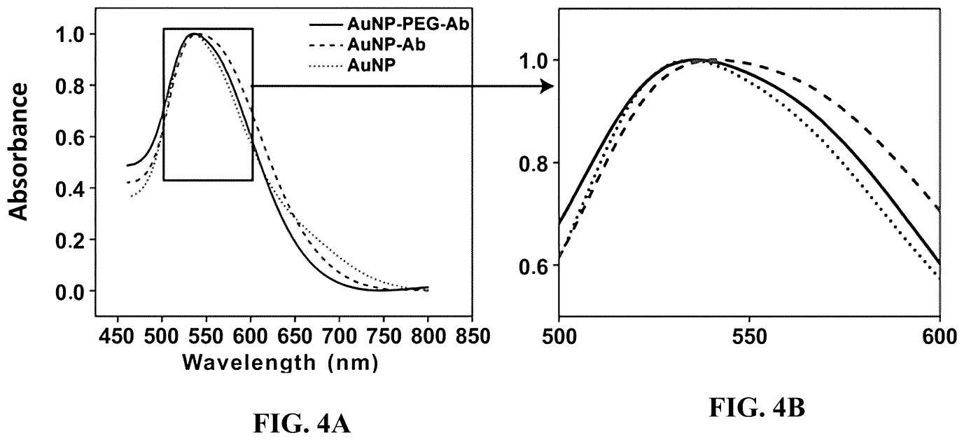

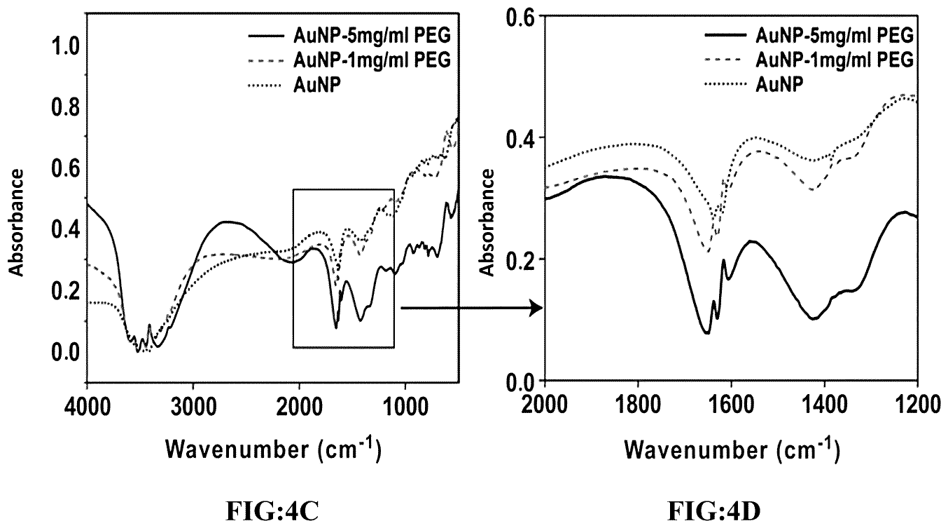

It can be seen from FIGS. 4A-4D that the absorption peak of AuNPs is at a wavelength of 534 nm, as shown in FIG. 4A and FIG. 4B, indicating the presence of AuNPs. The absorption peak of the AuNP-Ab conjugate is shifted from 534 nm to 541 nm, indicating that the detection antibody is successfully absorbed into the Au nanoparticles. The peak wavelength of AuNP-PEG-Ab is between those of AuNP and AuNP-Ab, because the addition of mPEG-SH, prevents the aggregation of AuNPs and makes the colloidal particles more dispersed. PEGylation reduces the hydrodynamic diameter from 86.64 nm to 71.90 nm, further improves the dispersion of Au-Ab, and obtains a blue shift of the UV-visible spectrum. The FTIR spectra of FIG. 4C and FIG. 4D further prove that PEG has been successfully conjugated into particles. The wavelength around 1650 cm⁻¹ is due to the addition of COO ions and PEG, many electron-withdrawing groups are shown, which makes the conjugate effect of COO stronger, so the second peak at 1600 cm⁻¹ is clearer.

The first preparation method for CL-LFIA includes the following steps:
a, 1.08 mg·mL⁻¹ cardiac troponin cTnI capture antibody is immobilized on nitrocellulose membrane 4 to form the test line, and then 1.5 mg·mL⁻¹ goat anti-mouse IgG antibodies is immobilized on nitrocellulose membrane 4 to form the control line;
b, the prepared the AuNP-Ab-HRP-PEG conjugate is dispensed onto conjugate pad 3;
c, sample pad 2, conjugate pad 3, nitrocellulose membrane 4, and absorbent pad 5 are overlapped to backing plate 1 in turn, and then the product is cut into strips with a width of 2 mm, and the strips are placed in a dry environment at room temperature for further use.

The detection method is as follows: the analyte is added to sample pad 2 or conjugate pad 3 during the detection, and then waiting for 0.5-10 minutes, a chemiluminescent substrate is added, and a photo is taken for recording after a luminescence.

The second preparation method for the CL-LFIA, in which the AuNP-Ab-HRP-PEG conjugate is not added during the preparation process, and other steps are the same as the first preparation method.

The detection method is as follows: the analyte is mixed with the AuNP-Ab-HRP-PEG conjugate during the detection, and then the mixture is dripped into sample pad 2 or conjugate pad 3, and then waiting for 0.5-10 minutes, and then a chemiluminescent substrate is added, a photo is taken for recording after a luminescence.

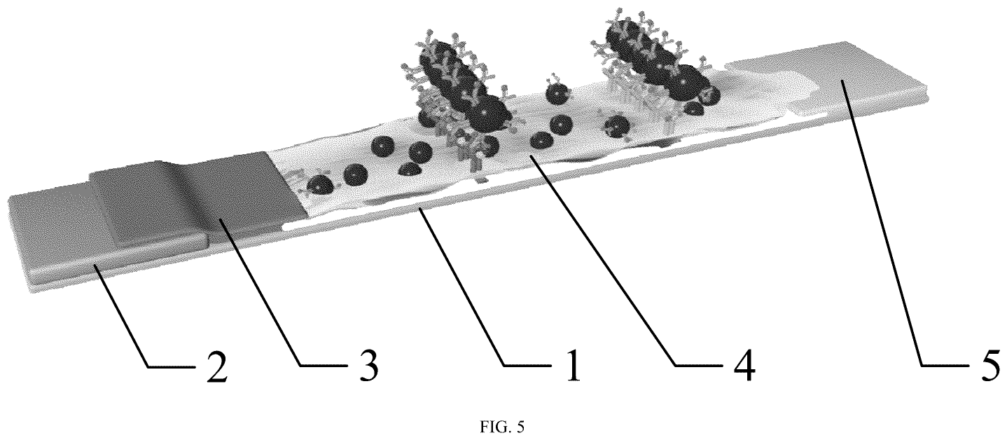

The AuNP-Ab-HRP-PEG conjugate is applied to a chemiluminescent lateral flow immunoassay strip prepared by first preparation method, as shown in FIG. 5. After the cardiac troponin cTnI is mixed with the AuNP-Ab-HRP-PEG conjugate, cardiac troponin cTnI will bind to the goat anti-mouse IgG antibody on the AuNP-Ab-HRP-PEG conjugate. The mixture is coated on a chemiluminescent lateral flow immunoassay strip, and the antigen will further immobilize to the immobilized antibody on the strip and form a sandwich structure on the test line. The additional AuNP-Ab-HRP-PEG conjugate is captured by goat anti-mouse IgG antibody on the control line. Finally, due to the red color of the gold nanoparticles, there are two red lines on the nitrocellulose membrane, indicating that the target is successfully detected.

Optimization of the CL-LFIA test is based on the strip prepared by the first preparation method.

Optimization of the CL-LFIA test: some basic parameters are evaluated and optimized to obtain the most intensive optical signal on the strip, including antibody concentration, PEG concentration, and detection time.

### (1) Optimization of Antibody Concentration:

First, different volumes (1,2,3,4 and 5 μL) of 1 mg·mL⁻¹ cTnI detection antibody are added of to 100 μL of AuNPs, and the other steps are the same as the preparation steps of the AuNP-Ab-HRP-PEG conjugates and strips. After the preparation is completed, the light intensity on the conjugate pad with different concentrations of antibodies is analyzed. Secondly, 2, 4, 6, 8, and 10 L of 50 mg·mL⁻¹ mPEG-SH are added to the AuNP solutions, and the light intensity on the strip is analyzed to optimize the concentration of mPEG-SH. Then, the mixing time of the analyte cTnI and the conjugate solution is optimized, 10 μL of 100 ng·mL⁻¹ cTnI solution is added to 10 μL conjugate solution, the mixture is mixed for 1, 2, 3, 5, 10, and 30 minutes, respectively. Finally, after adding the CL substrate to the nitrocellulose membrane, the intensity of the optical signal changed with the signal acquisition time on the test line is analyzed. The optical signal intensity is collected by a photomultiplier tube once per second.

### (2) Optimization of the PEG Concentration:

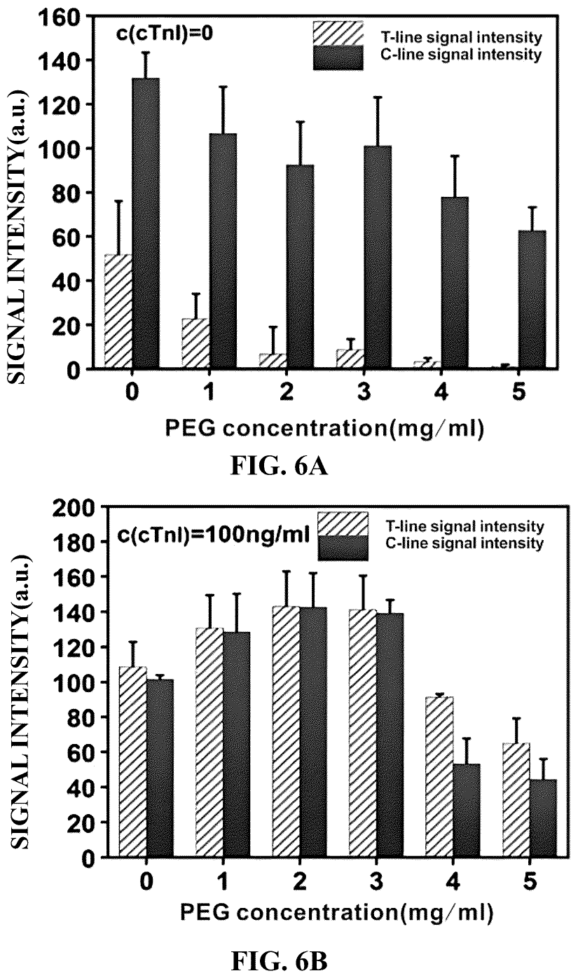

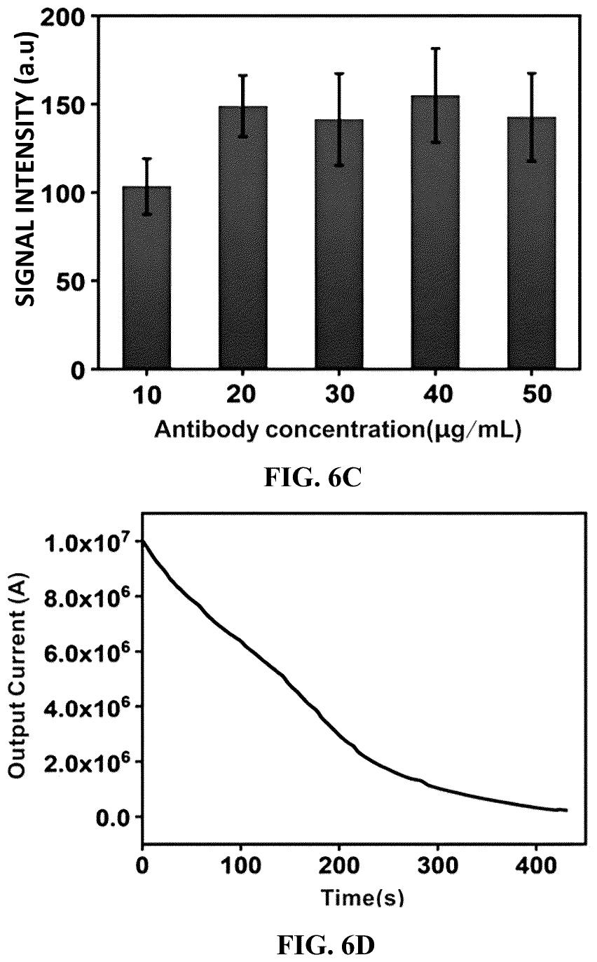

The effect of the PEG concentration on reducing the aggregation of the AuNPs in the CL-LFIA strips. As shown in FIG. 6A, in the absence of the analyte cTnI, the light intensity of the control line with different PEG concentrations is very large, while the light intensity of the test line is much smaller. The signal on the control line indicates that the strip is valid, while the signal on the test line indicates that there is a false positive signal on the test line. When the PEG concentration is 0, the optical signal at the test line is very strong, and the false positive is very obvious. When the PEG concentration is high enough, the false positive at the test line is weakened. As for the control line, the optical signal intensity is almost the same when the PEG concentration is 1-3 mg·mL⁻¹. However, when the concentration is higher than 3 mg·mL⁻¹, the large density of PEG molecules on the surface of the particles may mask the reaction site of the enzyme-catalyzed reaction and weaken the light intensity. In the absence of the analyte cTnI, when the concentration is higher than 3 mg·mL⁻¹, the light intensity on the control line decreases (as shown in FIG. 6A), which indicates that too high a PEG concentration may weaken the light signal intensity, too low a PEG concentration may lead to false positives on the detection line, and the concentration of PEG cannot be too high or too low. Similarly, the light intensity of the control line also showed the same trend for the detection of 100 ng·mL⁻¹ cTnI. When the PEG concentration is higher than 3 mg·mL-1, the light intensity of the control line and the detection line can be seen to decrease. According to the results of FIG. 6A and FIG. 6B, 4 mg·mL⁻¹ PEG concentration is an optimal concentration.

### (3) Optimization of the Detection Time:

The mixing time of AuNP-Ab-HRP-PEG conjugate and analyte cTnI is also optimized, and there is no significant difference between different mixing times (1,2,3,5,10, and 30 minutes). Therefore, once the analyte cTnI and the AuNP-Ab-HRP-PEG conjugate solution are fully mixed, the mixture can be added to the strip. Finally, after the addition of CL substrate, the concentration and detection time of HRP-conjugated detection antibody are optimized, as shown in FIG. 6C and FIG. 6D. The concentration is calculated by the number of micrograms of antibodies per milliliter of the AuNP solution, and the optimal antibody concentration is 40 μg·ml⁻¹. The lower antibody concentration will limit the HRP-catalyzed enzymatic reaction and weaken the detection signal. The concentration of the antibody should not be too high, which may lead to more HRP being absorbed on the nitrocellulose membrane, thus it may lead to high background noise signals. Therefore, the concentration of the antibody is optimized to 40 μg mL⁻¹ in the detection experiment. With the increase in detection time, the luminescence intensity on the strip gradually decreases. Therefore, the best detection time is to detect immediately after the addition of CL substrate.

Based on the optimization of the CL-LFIA test, the sensitivity and specificity of the LFIA strip are studied: In order to evaluate the sensitivity, 10 μL solution with a certain cTnI concentration (gradient setting from 0.01 to 100 ng·mL⁻¹) is added to 10 μL conjugate solution and mixing evenly, and then the mixed solution is placed on the strip. After 10 minutes, 50 μL deionized water is added to the strip to reduce the interference of background noise. After 10 minutes, 20 μL CL substrate solution is added to the nitrocellulose membrane, and the signal is obtained immediately through the smartphone camera in a dark environment. The exposure time is 3 seconds, and the image is analyzed by using ImageJ software. The specificity is related to the coefficient of variation, which is calculated by dividing the standard deviation (SD) by the average value. The y-axis detection limit (y-LOD) based on the y-axis blank limit (y-LOB) and the SD in the guidelines set by the Clinical and Laboratory Standards Institute are calculated: y-LOD=LOB+1.645×SD low concentration analyte, y-LOB=average blank+1.645×SD blank. From the y-LOD value, the concentration detection limit can be obtained from the calibration curve.

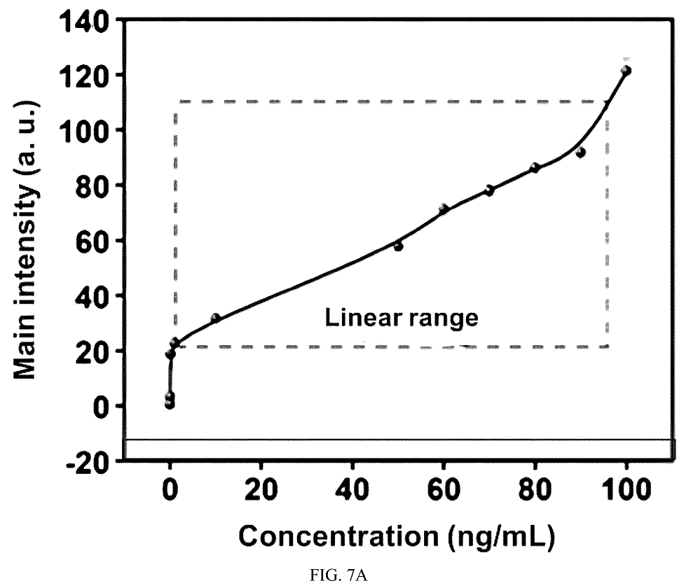

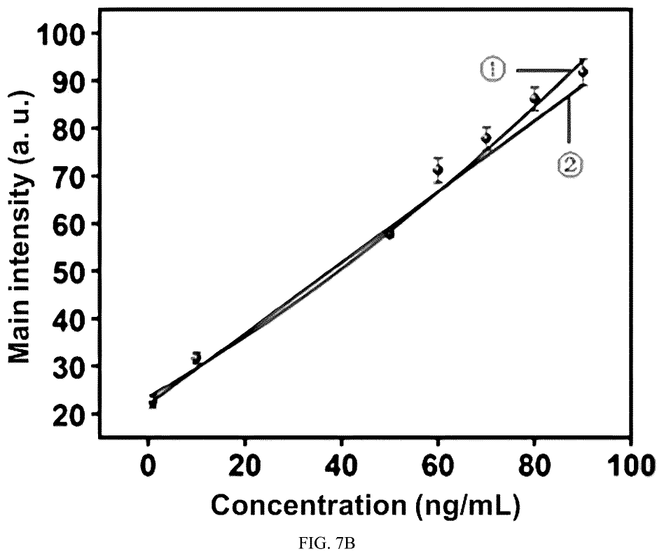

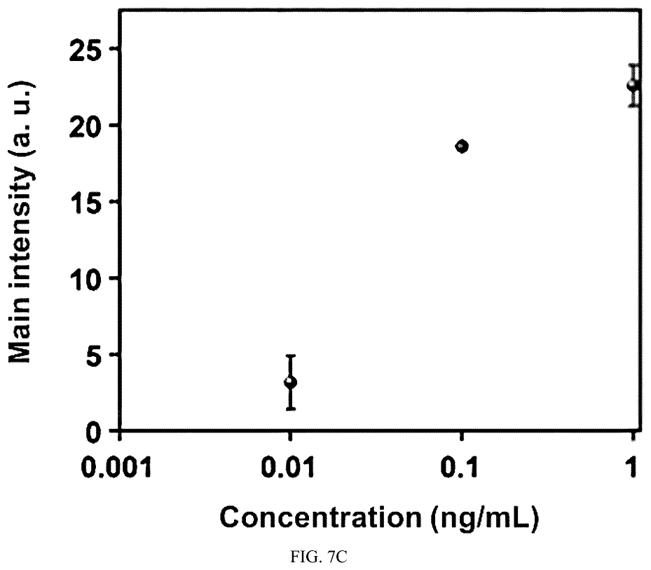

Based on the optimization of the CL-LFIA test, studying the determination of cTnI in PBS buffer solution: As shown in FIGS. 7A-7C, obtaining the linear range of the average intensity of the cTnI concentration of 10 pg·mL⁻¹-100 ng·mL⁻¹ as shown in FIG. 7A. The intensity of the test line is approximately proportional to the concentration range of cTnI, as shown in FIG. 7B, the data can be fitted to a polynomial for cTnI from 1 ng·mL⁻¹ to 90 ng·mL⁻¹, y=23.55-0.581x+0.00227x²(r²=0.993), as shown in line \textcircled{1} of FIG. 7B. A linear relationship can also be obtained for cTnI from 1 ng·mL⁻¹ to 90 ng·mL⁻¹, y=21.93+0.745x, r²=0.987, as shown in line \textcircled{2} in FIG. 7B. However, the polynomial model is more suitable than the linear model from the r-square data. Therefore, the polynomial model is used as the calibration curve. In addition, It can be seen from FIG. 7C that the detection limit of the concentration can reach 10 pg·mL⁻¹. The blank limit of the y-axis(y-LOB) of this method can be calculated by ‘LOB=average blank+1.645×SDblank’, which is determined to be 1.998 a.u., and y-LOD can be calculated by ‘LOD=L0B+1.645×SDblank’, which is determined to be 3.515 a.u. y-LOD and y-LOB both represent the CL intensity on the LFIA strip. From the calibration curve, it can be concluded that the detection limit of induced cTnI is about 0.01 ng·mL⁻¹. This method shows higher sensitivity. In addition, the error change from 0.1 ng·mL⁻¹ to 100 ng·mL⁻¹ is less than 10%, indicating the reliability and practical applicability of our method for detecting cTnI.

In the control group experiment without PEG, there is no significant difference in the detection of high concentrations of analytes. However, when detecting low concentrations of cTnI, a false positive error occurs, which greatly affects the results. When the concentration of cTnI is 0 or no more than 0.1 ng·mL⁻¹, the intensity of the test line is almost the same. Therefore, the control experiment can further illustrate that PEGylation of AuNPs can reduce false positive errors and improve the sensitivity and accuracy of LFIA.

Therefore, the invention adopts the above-mentioned chemiluminescent lateral flow immunoassay strip and its preparation method. The PEGylation of AuNPs can effectively reduce protein adsorption and nonspecific reaction, minimize cross reaction, and further improve the accuracy and sensitivity of detection.

Finally, it should be explained that the above embodiment is only used to explain the technical solution of the invention rather than restrict it. Although the invention is described in detail with reference to a better embodiment, the ordinary technical personnel in this field should understand that they can still modify or replace the technical solution of the invention, and these modifications or equivalent substitutions cannot make the modified technical solution out of the spirit and protection scope of the technical solution of the invention.

1. A chemiluminescent lateral flow immunoassay method, which is configured for reducing cross reactions and false positives in a detection of protein analytes, the method is not aimed at a diagnosis or a treatment of a disease, using an Au nanoparticle-antibody-horseradish peroxidase-polyethylene glycol (AuNP-Ab-HRP-PEG) conjugate to perform a lateral flow immunoassay on a strip for a detection of specific analytes;
a preparation of the AuNP-Ab-HRP-PEG conjugate comprises the following steps:
S1, conjugating a detection antibody of an HRP-conjugated analyte to AuNPs by physisorption method: adding a sodium carbonate solution to an AuNP solution, and then adding an HRP-labeled detection antibody, and then stirring gently on a rotator;
S2, linking an mPEG covalently to AuNPs by an Au—S bond: adding the mPEG solution to a mixture obtained in step S1 and stirring gently on the rotator for further incubation;
S3, incubating a BSA solution with the AuNPs and stirring gently on the rotator to block additional reaction sites on the AuNPs, then centrifuging an incubation product, and then adding a borate buffered solution of pH 8-9 to obtain the AuNP-Ab-HRP-PEG conjugate; and
S4, suspending the AuNP-Ab-HRP-PEG conjugate obtained by step S3 in a recovery solution containing 0.5-10% BSA, 1-10% sucrose, and 0.01-1% TWEEN® 20 borate buffered solution, and storing the conjugate for further use.

2. The chemiluminescent lateral flow immunoassay method according to claim 1, wherein the strip comprises a backing plate, a sample pad, a conjugate pad, a nitrocellulose membrane, and an absorbent pad; the sample pad, the conjugate pad, the nitrocellulose membrane, and the absorbent pad are arranged in turn to immobilize a surface of the backing plate; a surface of the nitrocellulose membrane is equipped with a test line and a control line, the test line is formed by immobilizing capture antibodies to the surface of the nitrocellulose membrane, the control line is formed by immobilizing IgG antibodies to the surface of the nitrocellulose membrane; the conjugate pad contains the AuNP-Ab-HRP-PEG conjugate.

3. The chemiluminescent lateral flow immunoassay method according to claim 2, wherein a preparation method for the strip comprises the following steps:
a, immobilizing the capture antibodies on the nitrocellulose membrane to form the test line, and then immobilizing the IgG antibodies on the nitrocellulose membrane to form the control line;
b, dispensing the prepared AuNP-Ab-HRP-PEG conjugate onto the conjugate pad;
c, overlapping the sample pad, the conjugate pad, the nitrocellulose membrane, and the absorbent pad to the backing plate in turn, and then cutting the backing plate into strips with a width of 1-10 mm, and placing the strips in a dry environment at room temperature for further use.

4. The chemiluminescent lateral flow immunoassay method according to claim 1,
wherein the chemiluminescent lateral flow immunoassay method comprises the following steps:
adding an analyte to a sample pad or a conjugate pad of the strip, the conjugate pad of the strip containing the AuNP-Ab-HRP-PEG conjugate, and then waiting for 0.5-10 minutes, adding a chemiluminescent substrate, taking a photo for recording after a luminescence.

5. The chemiluminescent lateral flow immunoassay method according to claim 1, wherein the strip comprises a backing plate, a sample pad, a conjugate pad, a nitrocellulose membrane, and an absorbent pad; the sample pad, the conjugate pad, the nitrocellulose membrane, and the absorbent pad are arranged in turn to be immobilized onto a surface of the backing plate; a surface of the nitrocellulose membrane is equipped with a test line and a control line, the test line is formed by immobilizing capture antibodies on the surface of the nitrocellulose membrane, the control line is formed by immobilizing IgG antibodies on the surface of the nitrocellulose membrane; the conjugate pad contains the AuNP-Ab-HRP-PEG conjugate.

6. The chemiluminescent lateral flow immunoassay method according to claim 5, wherein a preparation method for the strip comprises the following steps:
a, immobilizing the capture antibodies of an analyte on the nitrocellulose membrane to form the test line, and then immobilizing the IgG antibodies on the nitrocellulose membrane to form the control line;
b, overlapping the sample pad, the conjugate pad, the nitrocellulose membrane, and the absorbent pad to the backing plate in turn, and then cutting into strips with a width of 1-10 mm, and placing the strips in a dry environment at room temperature.

7. The chemiluminescent lateral flow immunoassay method according to claim 1, wherein the chemiluminescent lateral flow immunoassay method comprises the following steps:
mixing an analyte with the AuNP-Ab-HRP-PEG conjugate to form a mixed solution, and then adding the mixed solution to a sample pad or a conjugate pad of the strip, and then waiting for 0.5-10 minutes, adding a chemiluminescent substrate, taking a photo for recording after a luminescence.

8. The chemiluminescent lateral flow immunoassay method according to claim 2, wherein the chemiluminescent lateral flow immunoassay method comprises the following steps:
adding an analyte to the sample pad or the conjugate pad of the strip, the conjugate pad of the strip containing the AuNP-Ab-HRP-PEG conjugate, and then waiting for 0.5-10 minutes, adding a chemiluminescent substrate, taking a photo for recording after a luminescence.

9. The chemiluminescent lateral flow immunoassay method according to claim 3, wherein the chemiluminescent lateral flow immunoassay method comprises the following steps:
adding an analyte to the sample pad or the conjugate pad of the strip, the conjugate pad of the strip containing the AuNP-Ab-HRP-PEG conjugate, and then waiting for 0.5-10 minutes, adding a chemiluminescent substrate, taking a photo for recording after a luminescence.

10. The chemiluminescent lateral flow immunoassay method according to claim 5, wherein the chemiluminescent lateral flow immunoassay method comprises the following steps:
mixing an analyte with the AuNP-Ab-HRP-PEG conjugate to form a mixed solution, and then adding the mixed solution to the sample pad or the conjugate pad of the strip, and then waiting for 0.5-10 minutes, adding a chemiluminescent substrate, taking a photo for recording after a luminescence.

11. The chemiluminescent lateral flow immunoassay method according to claim 6, wherein the chemiluminescent lateral flow immunoassay method comprises the following steps:
mixing the analyte with the AuNP-Ab-HRP-PEG conjugate to form a mixed solution, and then adding the mixed solution to the sample pad or the conjugate pad of the strip, and then waiting for 0.5-10 minutes, adding a chemiluminescent substrate, taking a photo for recording after a luminescence.
